(19) United States
(12) Patent Application Publication
(10) Pub. No.: US 2003/0097148 A1
(43) Pub. Date: May 22, 2003

(54) Tissue fastener

(76) Inventors: Tero Valimaa, Tampere (FI); Pertti Tormala, Tampere (FI)

Correspondence Address: KENYON & KENYON, 1500 K STREET, N.W., SUITE 700, WASHINGTON, DC, 20005, US

(21) Appl. No.: 09/988,748

(22) Filed: Nov. 20, 2001

(51) Int. Cl. A61B 17/08

(52) U.S. Cl. 606/213

(57) ABSTRACT

The present invention provides a surgical fastener made of a bioabsorbable elastomeric material for the repair of tissues. The fastener may be elongated, thereby creating a compression force across or within the tissue being repaired. The present invention also includes embodiments drawn to methods of using the surgical fastener, such as applying the surgical fastener to a ruptured meniscus or using the surgical fastener to attach a fibrous implant or tissue transplant on or in a living tissue.

### FIELD OF THE INVENTION

The present invention relates to surgical fasteners and more specifically to bioabsorbable elastomeric surgical fasteners used to repair tissues, and methods of using these fasteners.

### BACKGROUND ART

With reference to the prior art in the field, it has been shown that fixation of meniscus traumas, like ruptures and lesions, by suturing with absorbable sutures gives better results than the removal of traumatized meniscal tissue (see, e.g., N. A Palmeri, T. F. Winters, A. E. Joiner and T. Evans, “The Development and Testing of the Arthroscopic Meniscal Staple”, Arthroscopy, Vol. 5, No. 2, 1989, p. 156 (Ref. 1)). However, arthroscopic suturing is a complicated and tedious technique where risks for the patient are significant because of the danger presented to vessels and nerves. Additionally, the suturing of meniscus ruptures leaves a single or several loops of sutures on the meniscal surface, which can irritate joint cavity tissues. Therefore, surgeons have long desired an absorbable meniscus lesion fixation device, like a staple or fastener, which has the advantages of absorbable suturing techniques but which can be used more rapidly and safely than sutures.

Several research groups have tried to develop absorbable meniscus lesion fixation devices such as clamps and tacks. However, the various demands upon such a device are high. It must be strong enough to maintain good contact of lesion tissues after the operation so that rapid healing occurs. The device must retain its strength long enough to allow for good healing. The rigidity of the device must be compatible with the tissue being repaired; if the rigidity of the device is too high it may cause tissue irritation and healing may be prolonged or even stopped. The device must be comprised of a material that is absorbed without causing complications that would prevent the healing of the lesion. Additionally, the installation of the device should be easy and rapid and should cause minimum operational trauma. Because of these high demands, efforts to develop an optimal absorbable meniscus lesion fixation device continue. Palmeri et al. reported in Ref. 1 the development of a method of meniscal repair using arthroscopically applied absorbable fasteners. However, the reported method was complicated because the final design used cannulation of the staple for needle-guided placement. Additionally, staple fracture, migration and articular abrasion were found.

In meniscal repair surgery, bioabsorbable devices are known in the art. For example, Schreiber (U.S. Pat. No. 4,873,976) discloses an arrow-like implant particularly intended for the surgical repair of meniscal ruptures. However, the arrow-like implant according to this publication has the disadvantage that particularly its proximal end (stem) contacts the outer surface of the meniscus and may cause tissue irritation and abrasion, particularly if left protruding from the outer surface of the meniscus. Similarly, Bays et al. (U.S. Pat. Nos. 4,884,572 and 4,895,141) and Winters (U.S. Pat. No. 5,059,206) describe a meniscal surgical-repair devices made of biodegradable material. A disadvantage of the device described in Bays is that the grip portion is bulky and may remain on meniscal surface causing irritation inside a joint cavity. In Winters, the fastener device has a proximal end that is bulky, comprising a cylindrical end, which protrudes partially above and/or below the outer surface of the meniscus, which may cause irritation and abrasion of the tissue.

Recent developments in bioabsorbable devices for use in surgical meniscal repair include a fastener and installation device disclosed by Justin et al. (U.S. Pat. No. 5,569,252) and Tamminmaki et al. (U.S. Pat. No. 5,562,704). In Justin, the fastener requires a rotating motion for installation that is slow and tedious to use arthroscopically. Additionally, rotating the fastener through fibrous tissue, such as meniscus tissue, has the risk that the fibrous tissue may twist around the turning implant, hindering or preventing the installation of the implant. Tamminmaki et al. (U.S. Pat. No. 5,562,704) discloses an arrow-like bioabsorbable implant wherein the proximal part of the implant (the wings) preferably remains on the surface of the meniscus. Therefore, there is a risk of irritation or abrasion of the opposite (femoral) cartilage surface if the surgical installation of the arrow is not performed precisely. Furthermore, if the proximal part with the wings is located inside of meniscal tissue, the surface capsule of the meniscus should first be cut horizontally with a special cutting blade. This lengthens the operation time and may damage the meniscus surface.

Lin et al. (U.S. Pat. No. 5,993,475) describes a fastener for repairing meniscal cartilage tears having a shaft and two ends, one end is adapted to pierce the tissue and the opposite end is adapted to fit a driver. The device has been subjected to a drawing operation after fabrication leaving the molecules in the shaft in a state of higher static residual stress. When the device is exposed to an aqueous environment, the water absorbed by the polymer lowers the glass transition temperature of the polymer. Lowering glass transition temperature allows increased molecular mobility causing the device to contract. The contraction pulls the torn tissues into close apposition during healing. The residual stress and aqueous environment are the key components in the action of this device. This implant, which needs aqueous environment, is depended on the amount of the tissue fluid in fixed tissue. The amount of tissue fluid varies in different tissues and therefore the action of this implant and the speed of contraction are limited by these tissue properties. Furthermore, the speed of the relaxation process that cause the contraction is slow and therefore the fixation result may not be seen during the surgery. The contraction force that is caused by relaxation is small and therefore the compression force between tissue lesions is low and the fixation may not be secure. The relaxation of the material in storage or in higher temperatures than normal room temperature, e.g. during transportation, may cause premature relaxation of static residual stresses and as a consequence the implant does not contract. Therefore, this implant is very sensitive to storage and transportation conditions.

Orthopedic and Musculoskeletal Markets Biotechnology and Tissue Engineering, Medical Data International, Inc., Irvine, Calif., USA, February 1997, p. 1-17 describes a bioabsorbable device for meniscal repair. This device has two legs with molded barbs that are attached by a flexible member composed of a resorbable suture. The device is installed into a meniscus with an arthroscopic tool so that the legs penetrate the rupture of meniscus to hold the edges together. However, the two-leg device requires a bulky installation tool, which makes arthroscopial installation of the device difficult.

Patent application PCT/EP 98/04183 describes a fastener for body tissue repair comprising a shaft comprised of a proximal portion, having an upper surface and a lower surface with first protrusions, and a distal portion, said distal portion having a sharpened tip and one or more first protrusions. The first protrusions of the fastener have proximal surfaces configured to arrest the movement of the shaft in the proximal direction and distal surfaces configured to permit the movement of the shaft in the distal direction. Further, the said proximal portion have second protrusions on the upper surface and lower surface of the proximal portion, wherein said second protrusions have distal surfaces configured to arrest the movement of the shaft in the distal direction. Although this implant sinks totally inside a tissue, such as a knee meniscus, the second protrusions can be damaged during the insertion of the implant into tissue or the second protrusions can damage the tissue, such as cutting the horizontal collagen fibers of the tissue, during the insertion.

A need therefore exists for bioabsorbable fastener, which provides stronger and safer fixation of tissue tears. The fastener must also be rapidly and easily installed and cause minimal trauma when installed.

### SUMMARY OF THE INVENTION

The present invention provides a bioabsorbable elastomeric surgical fastener for tissue repair. The surgical fastener forges a compression force between lesion tissues and secures fixation.

It is a further object to provide such a fastener that is rapidly and easily installed, provides strong and safe fixation of the tissue tear, implant or transplant, is minimally traumatic, and may be made from an elastomeric, nontoxic, biocompatible bioabsorbable polymer, polymer alloy or fiber reinforced polymer composite, specially designed to maintain its structural integrity during the healing of the tear and to prevent tissue abrasion.

It is a further object to provide a fastener which will be shot or pushed totally or for the most part inside of soft or tough tissue, like meniscal tissue, to penetrate the tissue (meniscal) tear and to hold the ruptured edges together and to cause a minimal trauma to the tissue through which the fastener passes.

In a preferred embodiment the elastomeric behavior of the fastener creates a compression force between tissue lesion sides. This compression force helps to keep the sides together, accelerates the healing, and assures secure fixation during the healing period. The elastomeric behavior of the fastener is also insensitive to environmental conditions, e.g., amount of tissue fluids or temperature; therefore it works in tissues that have a low amount of tissue fluids.

The present invention also includes embodiments drawn to methods of using the bioabsorbable elastomeric surgical fastener. In such an embodiment of the present invention, the method includes applying the bioabsorbable elastomeric surgical fastener to a ruptured meniscus. Another embodiment of the present invention includes using the bioabsorbable elastomeric surgical fastener to attach a fibrous implant or tissue transplant to living tissue.

### BRIEF DESCRIPTION OF THE DRAWINGS

FIGS. 1A-H illustrate side views of devices that are embodiments of the present invention.

FIG. 2 illustrates side views of different proximal and distal end profiles of devices that are embodiments of the present invention.

FIGS. 3A-D illustrate side views of ridged and camiulated devices that are embodiments of the present invention.

FIGS. 4-7 illustrate different ridge profiles in devices that are embodiments of the present invention.

FIGS. 8A-G illustrate sectional views of the installation of a device of the present invention into the torn meniscus.

FIGS. 9A-G illustrate sectional views of the installation of a device of the invention into a torn meniscus.

FIGS. 10A-G illustrate sectional views of the installation of a device of the invention into a torn meniscus.

FIG. 11 illustrates a sectional view of the fibrous structure of the meniscus.

FIGS. 12A-C illustrate sectional views of the fibrous structure of the meniscus in which a surgical device according to the present invention has been installed.

FIGS. 13A-B illustrate the fixation of a fibrous mesh to the surface of living tissue by means of devices that are embodiments of the present invention.

### DETAILED DESCRIPTION OF THE PREFERRED EMBODIMENTS

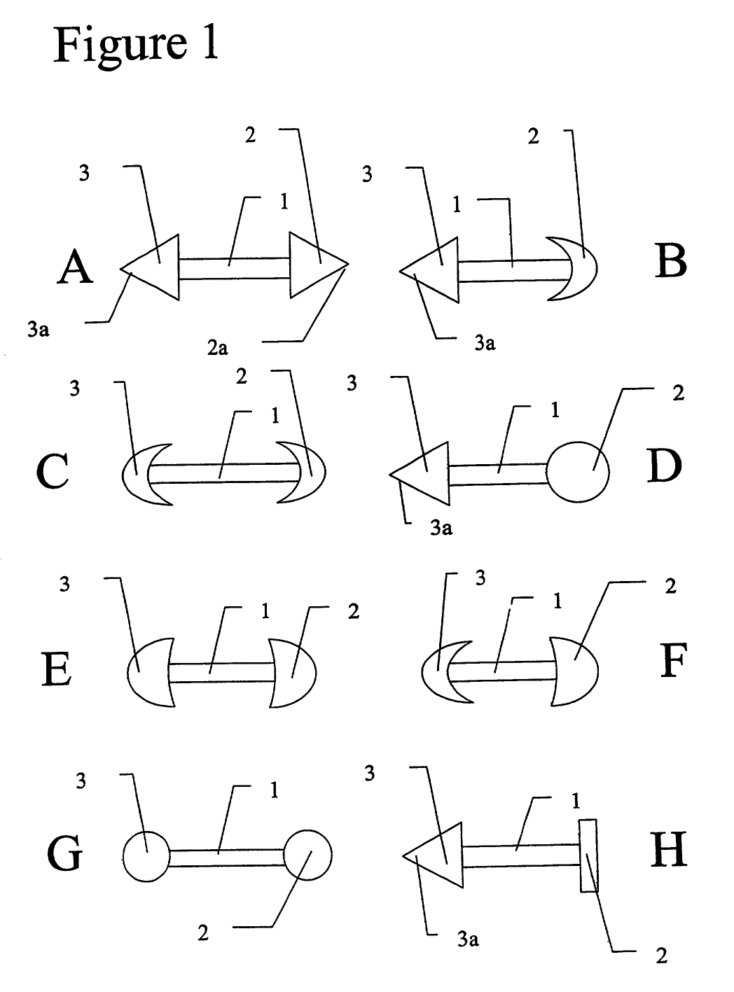

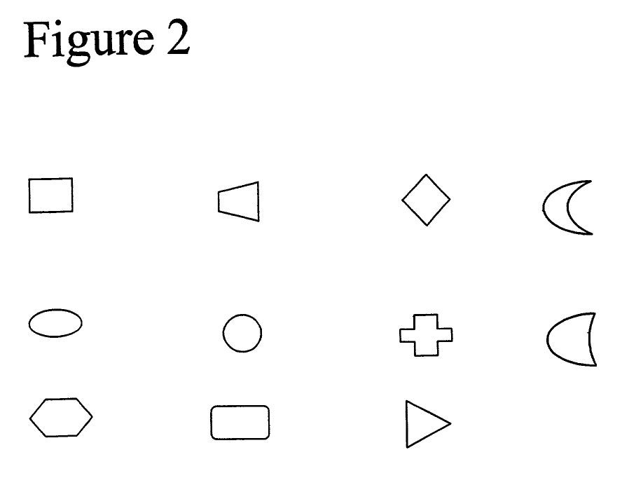

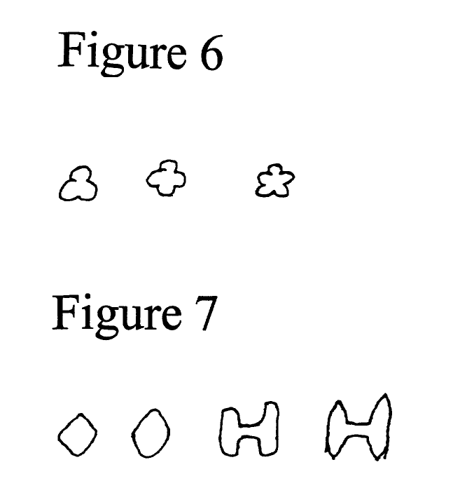

A description of the preferred embodiments of the present invention is presented with reference to FIGS. 1-12.

A preferred exemplary embodiment of the present invention comprises a fastener and method for repairing a soft or tough tissue, like a knee meniscal tear, in a human patient.

FIGS. 1A-H illustrate, as viewed from the side, preferred embodiments of the fastener. It is designed to have an arrow or rod shape, and it comprises a shaft 1, whose proximal portion is formed with a end 2 for the purpose of providing the locking element to lock the fastener 1 in relation to the meniscus with the proximal end 2 remaining partially on the surface or totally inside of the meniscus, and whose distal portion is formed with a head 3 with a sharp or blunt tip. A purpose of the proximal end 2 is to stop the implant inside of the meniscus, the proximal end remaining partially on the surface of meniscus or totally within the meniscus during the final stage of the installation of device, and to prevent further sinking of the device into the meniscus, when the implantation is complete and the device is compressing the tissue into which it has been inserted. Using a proper insertion device, the fastener is forced inside of the meniscus or at least so deep into the meniscus that the end 2 is located at the bottom of a small notch on the surface of meniscus, thus causing no disturbance to the opposite joint cartilage surface of the distal joint surface of femur.

FIGS. 1A-H illustrate some of the various possible geometries for the ends 2 and 3. The fastener of FIG. 1A has relatively sharp tips 2a and 3a, while FIG. 1G describes a fastener with ends that are more blunt. FIG. 1D describes a fastener with both a blunt and a sharp end.

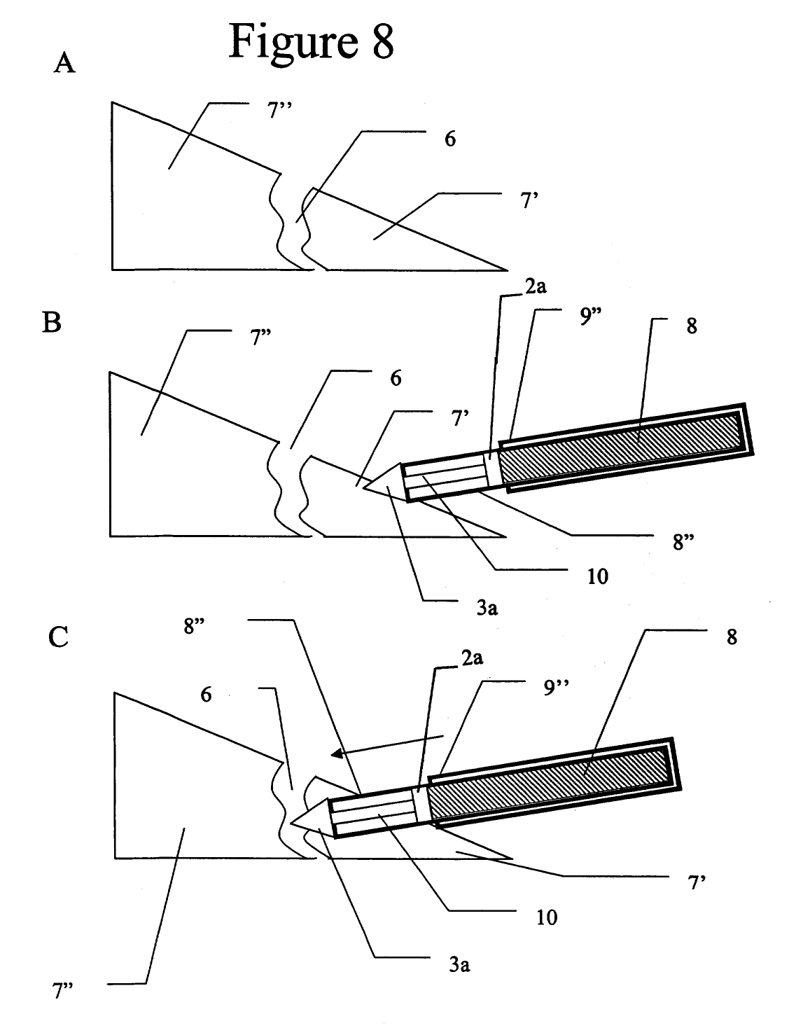

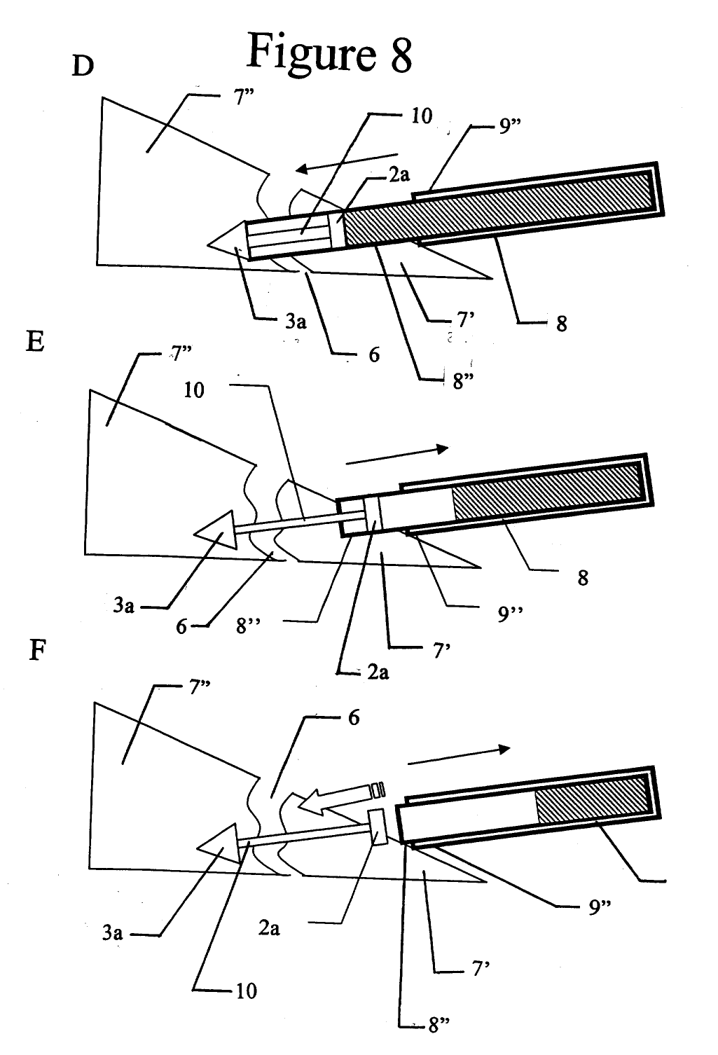

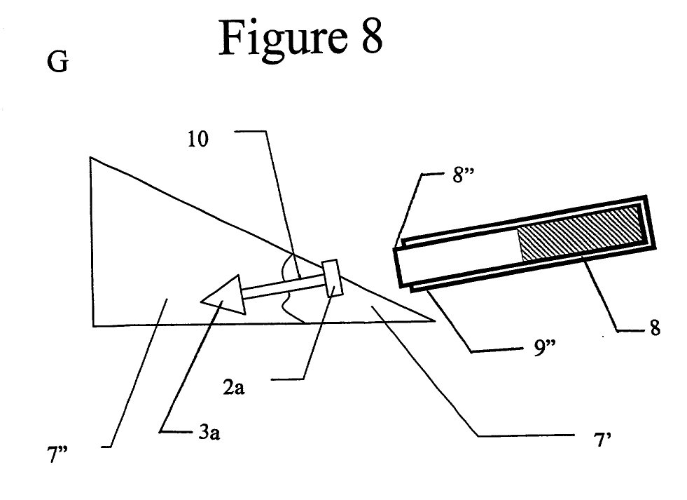

The proximal and distal ends 2 and 3 effectively lock the device within or partially within the meniscus, preventing its movement both in the direction of installation and in the direction opposite to it. After the implant has been released in the meniscus, the elastomeric behavior of the material compresses the rupture surface as shown in FIGS. 8A-G and 9A-G. This occurs because the ends 2 and 3 lock in and push the sides of the rupture against each other during the final phase of installation as shown in FIGS. 8A-G and 9A-G.

Because the device, preferably, is located mainly or totally inside of the meniscus, leaving at most only a small flexible prominence on the meniscus surface, the risks of prior art devices, such as complications originating (a) from the presence of the rigid bulky proximal part of the device on the meniscal surface, or (b) from the uncertain fixation of the meniscus lesion by only the protrusions, notches or threads, are eliminated.

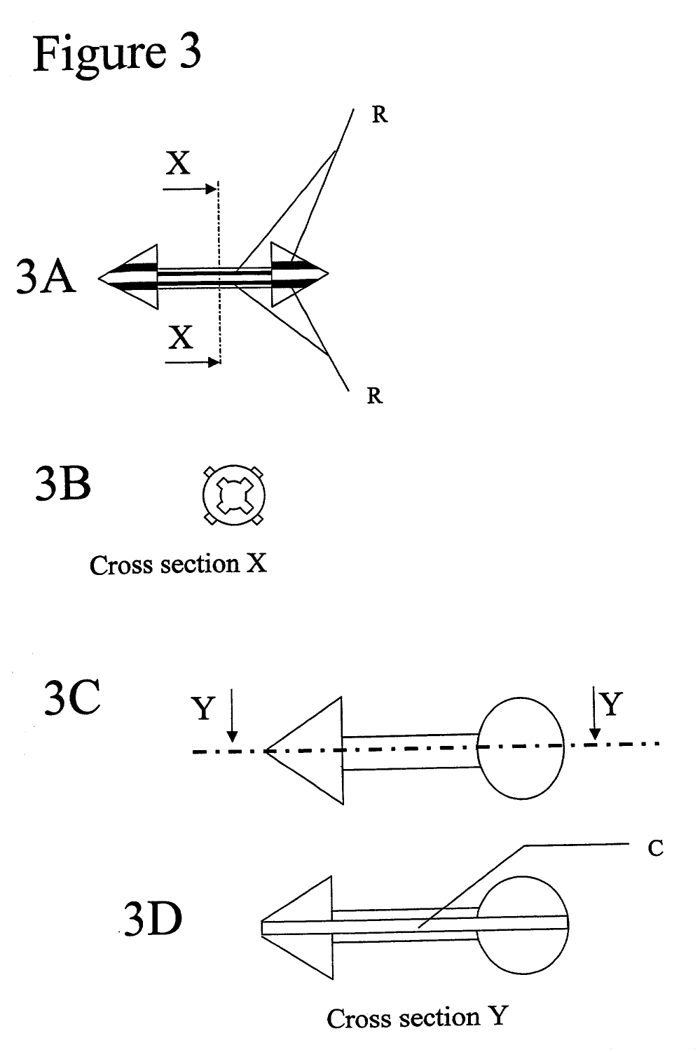

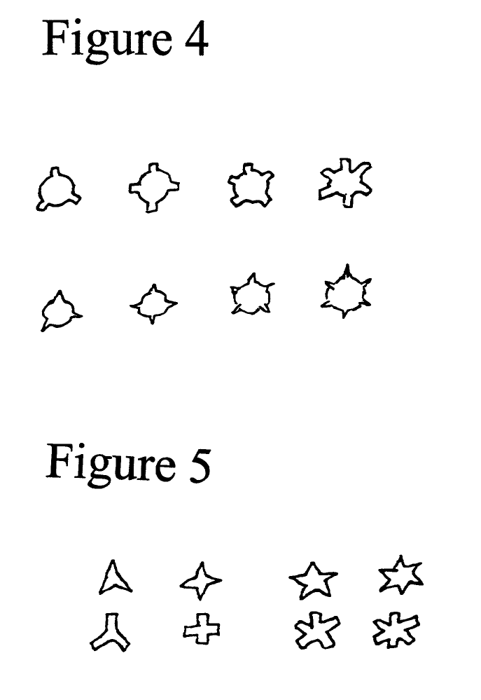

The surface of the fastener can also be machined or molded to include longitudinal ridges. FIG. 3A shows a side-view perspective of such a fastener having on its surface longitudinal ridges (R), which are arranged onto the surface of the fastener according to FIG. 3B, which gives the cross section of the fastener in the plane X-X of FIG. 3A. The longitudinal ridges on the surface of the fastener can be arranged in a number of possible geometries. Additionally, the geometry of the ridges can be varied to influence the gripping capacity of protrusions and of the tapered proximal end on meniscal or other tissue. FIGS. 4-7 illustrate some preferred embodiments of the cross-sectional structures of ridged fasteners of the present invention.

The bioabsorbable implants of this invention can be manufactured of elastomeric bioabsorbable polymers, copolymers or polymer mixtures or alloys with crosslinking or molding methods known in the prior art, see, e.g. D. W. Grijpma et al., Polymer, 34 (1993) 1496-1503. It is also possible to use the techniques described in U.S. Pat. No. 4,743,257 to mold in a compression or injection mold elastomeric absorbable fibers and binding polymer together to create a fiber-reinforced or especially a self-reinforced structure. The implants of this invention can be molded in a single compression molding cycle, or the protrusions can be machined on the surface of a fastener after an initial molding cycle.

An oriented and/or self-reinforced structure can also be created during extrusion or injection molding of absorbable polymeric melt trough a suitable die or into a suitable mold at high speed and pressure. When cooling occurs at suitable conditions, the flow orientation of the melt remains in the solid material as an oriented or self-reinforcing structure. In an advantageous embodiment, the mold can have the form of the implant, but it is also possible to manufacture the implants of the invention by machining (possibly using heat) and thermoforming (e.g. by bending the proximal end) injection-molded or extruded semi-finished products.

Oriented and/or self-reinforced or otherwise fiber-reinforced implants of this invention can be manufactured by molding the reinforcement fiber-polymer matrix to the final product in a mold, whose mold cavity has the form of the final product. Alternatively, the final form can be machined mechanically (possibly also using heat) on a pre-form, such as a melt-molded and solid-state drawn rod, as is described, e.g., in U.S. Pat. No. 4,968,317.

In some advantageous embodiments of this invention, the orientation and/or reinforcing elements of the elastomeric self-reinforced structure are mainly oriented in the direction of the long axis of the shaft of the implant and also into the tapered, curved proximal end. The reinforcement elements may extend into any protrusions or ridges of the implant. The reinforcement elements can also turn spirally around the long axis of the implant and also into the wider ends. Further, other different orientations of reinforcement elements in elongated samples, which are familiar from composite technology, can be applied to the present invention. A general feature of orientation and/or fiber-reinforcement or self-reinforcement of the implants of this invention is that many of the reinforcing elements are oriented in such a way that they can effectively carry the different external loads (such as tensile, bending and shear loads) that are directed to the healing rupture (for example loads to a meniscus caused by the movements of the patient's knee).

According to an advantageous embodiment of the invention, the meniscal repair implant, or a special coating layer on its surface, may contain one or more bioactive substances, such as bioabsorbable ceramic or glass particles, antibiotics, chemotherapeutic substances, angiogenic growth factors, substances accelerating the healing of the wound, growth hormones and the like. Such bioactive meniscal repair implants are especially advantageous in surgical use, because they chemically contribute to the healing of the lesion in addition to providing mechanical support.

The fastener can also include cannulation, which can be machined or molded. FIG. 3C shows a side-view perspective of such a fastener having a longitudinal cannulation (C), as shown passing through the fastener according to FIG. 3D, which gives the cross section of the fastener in the plane Y-Y of FIG. 3C.

FIGS. 8A-G, 9A-9G and 10A-G illustrate various preferred methods for installing certain preferred embodiments of fasteners of the invention into ruptured meniscal tissue. FIG. 8A illustrates, as viewed from the side, a meniscus with a rupture 6 separating the meniscus into a proximal side 7′ and a distal side 7″. As seen in FIGS. 8B-D, during the operation the tip 8″ of an installation cannula 8 and tapered tip 3 a of the fastener 10 are pushed into the knee joint through a small incision and then across the meniscal tear. The tip of the outer tube 9″ is located on the surface of the proximal part of the meniscus 7′.

As seen in FIG. 8E, the installation cannula 8 moves to the right (proximally) and elongates the fastener 10 inside the cannula 8. The cannula 8 can be accelerated to a high speed so that the elongation process occurs rapidly and fixes the tear 6 at a high speed, as is illustrated in FIGS. 8D-8G. The tip of the outer tube 9″ stays on the surface of the meniscus until the fastener has been released from the cannula tip 8″. The cannula 8 stops at the final stage of its movement by way of, e.g., a stopper (not shown) at the proximal end of the cannula 8. Typically, the tip of the cannula 8 stops when 0.5-1 mm inside the tip of outer tube 9″. Then the elongated fastener is released and the proximal end 2a of the fastener becomes located at the bottom of a small notch formed on the surface of the meniscus as the fastener attempts to contract. When the location of the cannula tip 8″ on the meniscal surface is selected in a proper way, typically 2-4 mm at the front of the meniscal tear 6, and the direction of the cannula is proper, the cannula with a fastener penetrates the proximal meniscus part 7′ and the tear plane 6 and the fastener closes the tear with the compression force created by the elongation of the fastener's elastomeric material. As FIGS. 8F and 8G schematically show, when the elongated fastener is released, it creates a compression force across the rupture 6, as it attempts to contract thereby helping to close the rupture 6. Accordingly, the rupture 6 is closed effectively, the fastener is locked in to its position to keep the rupture 6 closed and at most only a small, soft part of the whole fastener is left on meniscal tissue.

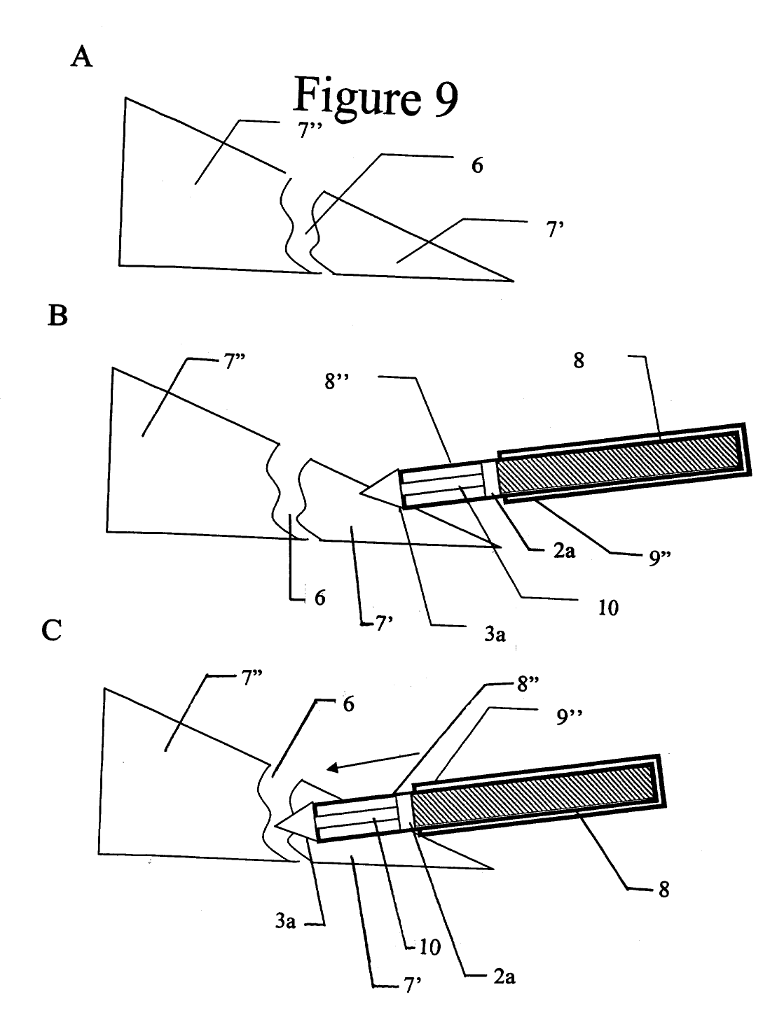

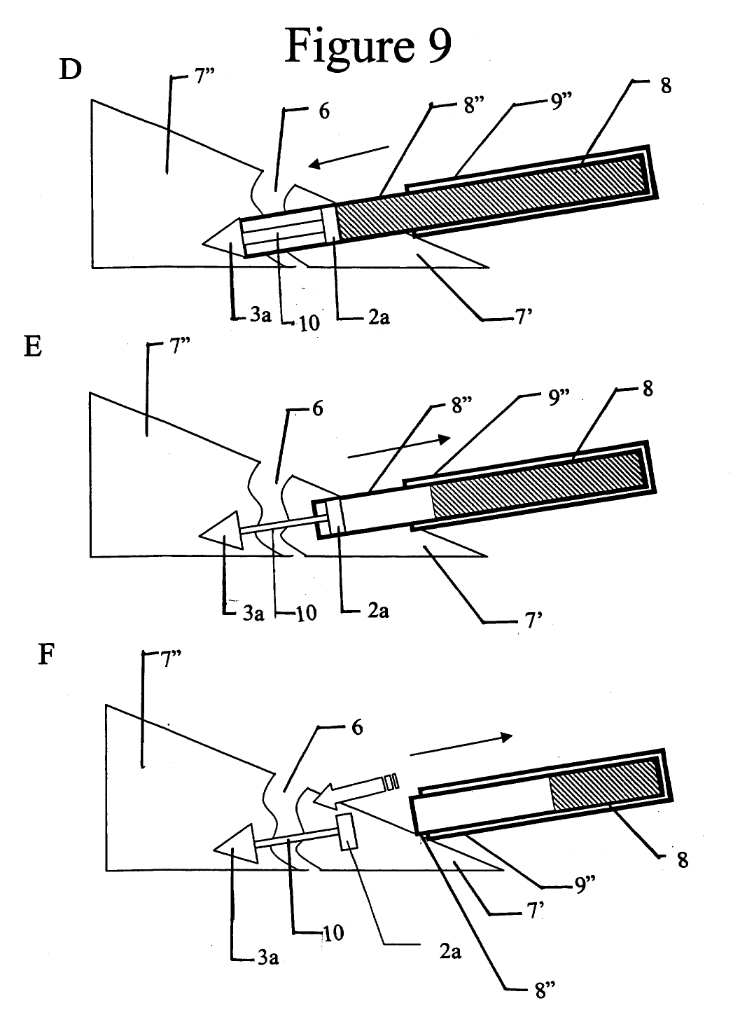

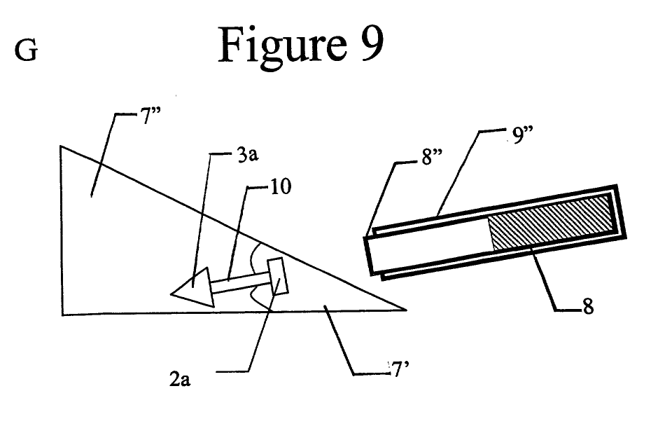

As seen in FIGS. 9A-9G, in another preferred method of insertion, the fastener 10 can be inserted so that it is located entirely within the meniscus. This insertion technique is similar to that shown in FIGS. 8A-8G, however as seen particularly in FIGS. 9E-9G, the positioning and length of the cannula 8 can be altered so that, when the fastener 10 is elongated and released, the proximal end 2a of the fastener 10 is located under the surface of the meniscus. As seen in FIG. 9G, because of the elastomeric nature of the fastener 10, it attempts to contract after being elongated and released, and this contraction force helps to pull the meniscal rupture 6 closed.

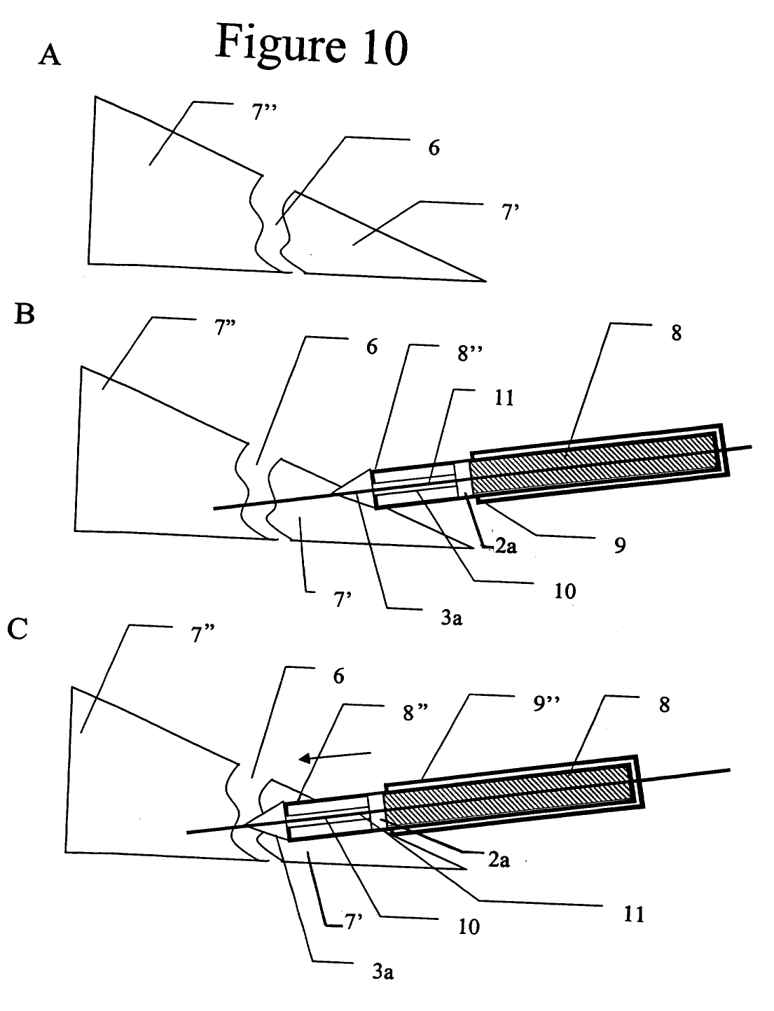

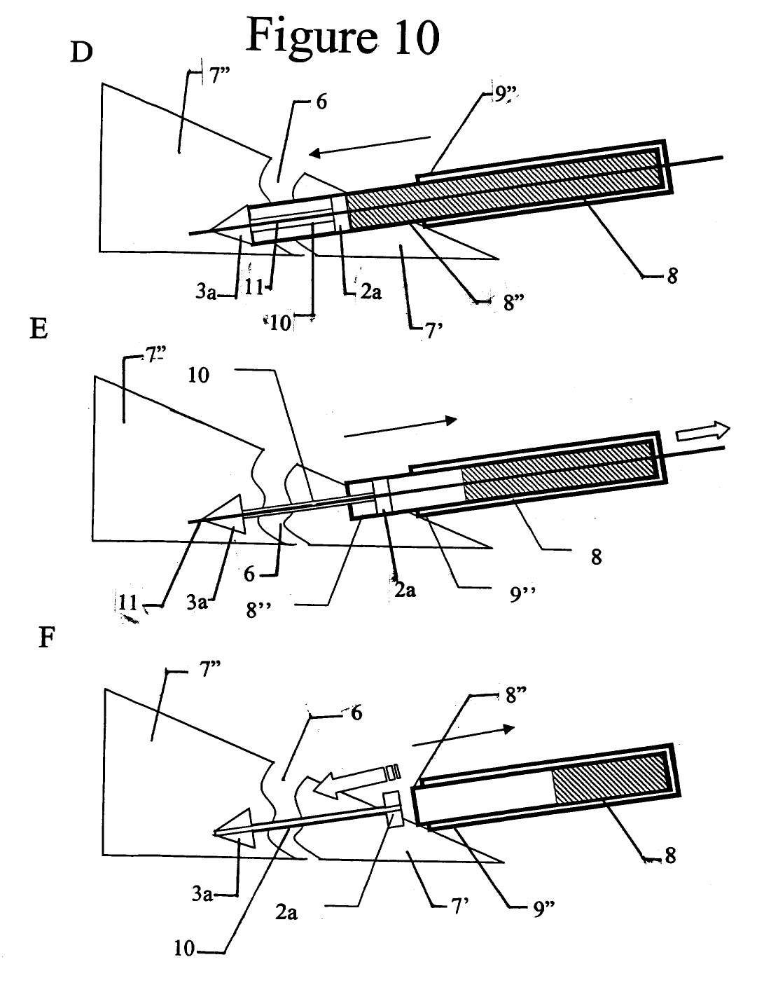

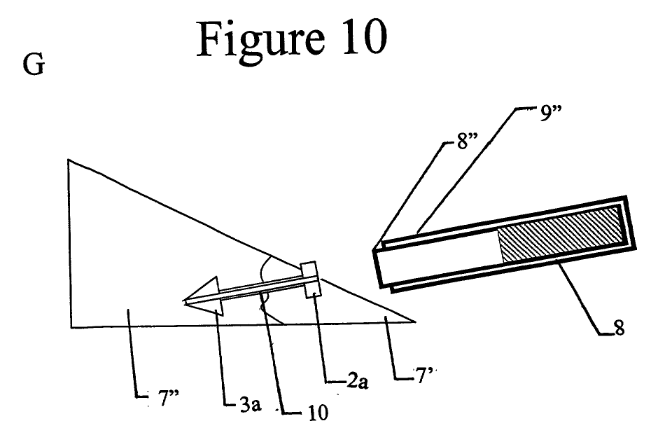

FIGS. 10A-G illustrates a preferred method for installing fasteners of the invention into ruptured meniscal tissue using a cannulated fastener and guiding wire 1. FIG. 10A illustrates a meniscus with a rupture 6 separating the meniscus into a proximal side 7′ and a distal side 7″. As seen in FIGS. 10B-10D, during the operation, first the guiding wire 11 is pushed into the knee joint through a small incision and across the meniscal tear followed by the tip 8″ of an installation cannula 8 and the tapered tip 3a of the fastener 10. The tip of the outer tube 9″ is located on the surface of the proximal part of the meniscus 7′. The guide wire passes across the tear and helps to prevent lateral movement of the meniscus when the fastener and the installation cannula are pushed across the rupture. Otherwise, as seen in FIGS. 10E-G, this insertion technique is similar to those shown in FIGS. 8 and 9.

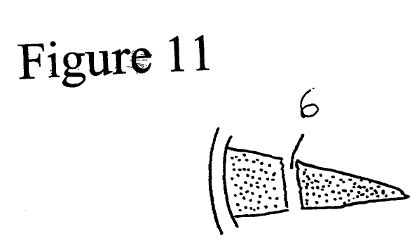

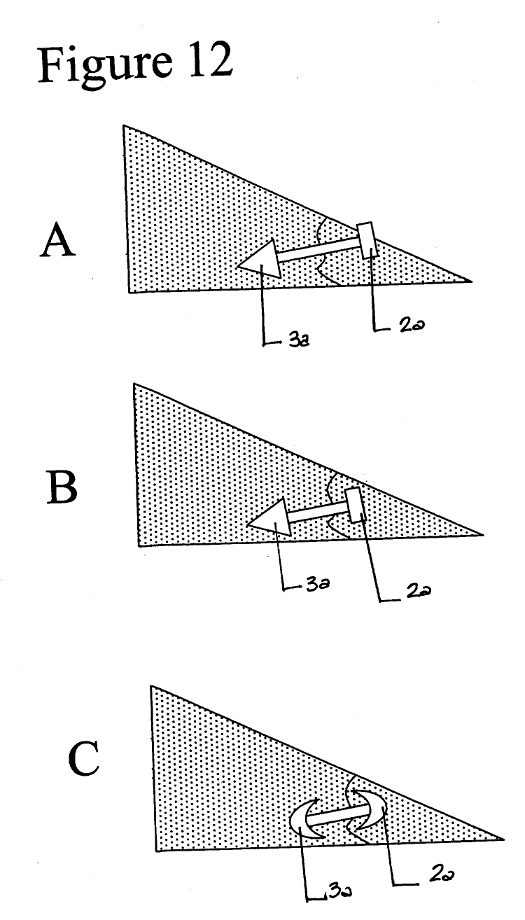

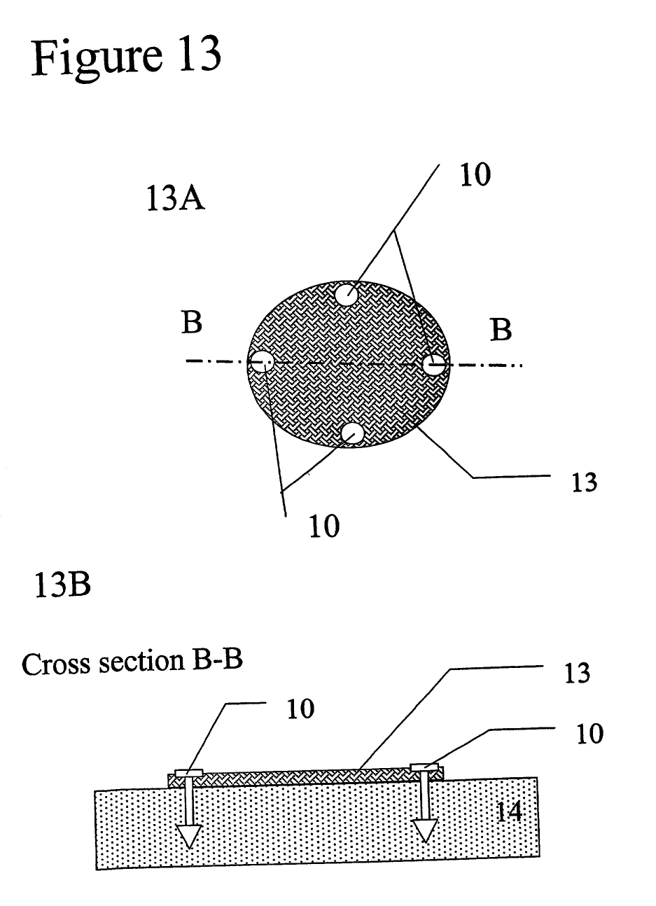

It is typical that the microstructure of a meniscus contains reinforcing collagen fibers. Inside the meniscus, many collagen fibers are oriented in a horizontal plane nearly parallel to the lower surface of the meniscus. The cut ends of horizontal collagen fibers, when examined in a cut cross-section of a meniscus, can be seen microscopically as points on the cross-sectional surface, as shown in FIGS. 11 and 12. The typical vertical meniscus lesion (rupture) 6 develops along the long axes of collagen fibers, because of the relatively weak binding forces between collagen fibers.

Because of the arrangement of the majority of the reinforcing horizontal collagen fibers inside of the meniscus, as FIGS. 11 and 12 schematically show, it is preferable that the distal end 3a and proximal end 2a of the fastener of the present invention protrude at least on the upper and/or lower surfaces of the fasteners. Thus, as the fastener penetrates into the meniscal tissue, the distal end slides forward through the collagen fiber bundles and grabs between the horizontal collagen fiber bundles, locking the fastener in place. Further, it can be advantageous if the proximal portion of the fastener is wide so that the end compresses the meniscal rupture and thus locks the proximal portion to its place when the elongated fastener is released by the installation device. This is schematically shown in the meniscal cross section of FIGS. 12A-C.

In addition to using the fasteners of this invention to repair tears in living tissues, these fasteners can be used to attach synthetic fibrous implants, like membranes, meshes, non-woven felts, fibrous scaffolds, etc., onto or into living tissues. Such synthetic fibrous implants are described, e.g., in EPO Pat. No. 0423155, U.S. Pat. No. 6,007,580 and PCT/EP 98/03030.

In a preferred embodiment in which the fasteners of this invention are used to attach a synthetic fibrous implant onto or into living tissue, the fibrous implant is first aligned on the surface or inside of living tissue. Thereafter, fasteners are pushed one after another through the synthetic implant so that the distal part of fastener locks the fastener into the tissue below the synthetic implant and the proximal end of the implant remains on the synthetic implant, securing it on the surface (or inside of the living tissue). FIG. 1 3A shows, as seen from above, and 13B, as a side view of plane B-B, a fibrous mesh 13 secured with fasteners 10 onto a living tissue 14.

The fasteners of this invention also can be used for attaching living tissue transplants such as autografts, allografts and xenografts, like collagen membranes and felts, periosteum transplant or connective tissue transplant.

The implants of the present invention may be sterilized by any of the well-known sterilization techniques, depending on the type of material used in manufacture of the implant. Suitable sterilization techniques include heat or steam sterilization, radiation sterilization such as cobalt 60 irradiation or electron beams, ethylene oxide sterilization, plasma sterilization and the like.

Having described the preferred embodiment of an improved surgical fastener and method of deploying the same in accordance with the present invention, it is believed that other modification, variations and changes will be suggested to those skilled in the art in view of the teachings set forth herein. It is therefore to be understood that all such variations, modifications and changes are believed to fall within the scope of the present invention as defined by the appended claims.

We claim:

1. A surgical fastener for repairing tissue, said fastener comprising:
a shaft, said shaft having a longitudinal axis and proximal and distal portions, said proximal portion comprising at least one protrusion configured to resist movement in the distal direction,
said distal portion comprising at least on protrusion configure to resist movement in the proximal direction,
said shaft comprising an elastomeric polymer.

2. The surgical fastener of claim 1, wherein said distal portion comprises a tapered end.

3. A surgical fastener of claim 1, wherein at least one of said proximal protrusions protrudes farther from said shaft than said distal protrusions.

4. A surgical fastener according to claim 1, wherein the shaft comprises one or more longitudinal ridges, wherein the one or more longitudinal ridges are positioned between the distal and proximal portions of the shaft.

5. A surgical fastener of claim 2, wherein the shaft comprises one or more longitudinal ridges along said distal portions, wherein said distal protrusions protrude from said one or more longitudinal ridges.

6. A surgical fastener according to claim 1, comprising one or more longitudinal cannulation holes.

7. A surgical fastener according to claim 1 wherein the fastener comprises a bioactive material.

8. A method of repairing a ruptured meniscus, comprising the steps of:
aligning the fastener of claim 1 on the surface of the meniscus;
positioning the fastener into the meniscus so that the fastener bridges the rupture;
elongating the fastener to create a compression force within the fastener; and
releasing the elongated fastener such that the compression force tends to close the rupture.

9. The method of claim 8, wherein, following insertion, at least a part of the proximal portion of the fastener is at the surface of the meniscus.

10. The method of claim 8, wherein, following insertion, the fastener in its entirety is located within the meniscus.

11. The method of claim 8, wherein the fastener is elongated prior to being positioned across the rupture.

12. A method of attaching an implant or tissue transplant to tissue comprising the steps of:
aligning the implant transplant on the surface of the tissue;
positioning the fastener of claim 1 so that it passes through the implant or transplant and into the tissue such that at least a part of the distal portion of the fastener is located in the tissue and at least a part of the proximal portion of the implant is positioned on the surface of the implant or transplant;
elongating the fastener to create a compression force within the fastener; and
releasing the fastener such that the compression force tends to keep the implant or tissue transplant on the tissue.

13. A method according to claim 12, where the implant or transplant is selected from the group comprising a synthetic, polymeric mesh or scaffold, collagenous mesh or scaffold, periosteum transplant and connective tissue transplant.
